(19) United States
(12) Patent Application Publication
(10) Pub. No.: US 2003/0105441 A1
(43) Pub. Date: Jun. 5, 2003

(54) Absorbent article comprising polymer with low gel integrity index

(76) Inventor: Harry J. Chmielewski, Brunswick, GA (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(21) Appl. No.: 09/996,826

(22) Filed: Nov. 30, 2001

(51) Int. Cl. A61F 13/15

(52) U.S. Cl. 604/368

(57) ABSTRACT

Absorbent articles, such as diapers, incontinence products, training pants, sanitary napkins, and the like, providing unexpectedly superior absorbency and methods of preparing the absorbent articles are disclosed. The absorbent articles comprise an absorbent core comprising a superabsorbent polymer having a Gel Integrity Index (GII) of less than about 500 kg mm.

### FIELD OF THE INVENTION

The present invention relates generally to an absorbent composition for absorbent articles such as diapers, incontinence products, training pants, sanitary napkins, and the like. In particular, the present invention is directed to absorbent articles, having unexpectedly superior absorbent properties, comprising an absorbent core disposed between the substantially impermeable backsheet and the permeable topsheet, said absorbent core comprising a polymer having a Gel Integrity Index (GII) of less than about 500 kg mm.

### BACKGROUND OF THE INVENTION

Disposable absorbent articles typically include a moisture-impervious backing sheet, an absorbent pad, and a liner sheet that contacts the body of a person wearing the article. In addition, elasticized regions are provided around the edges of the article to secure the article about the waist and legs of a wearer. Diapers typically further comprise opposed front and rear waist portions defining a waist opening, a crotch portion disposed there between, and a pair of elastically contractible leg openings along the side edges of the crotch portion. Disposable diapers having elasticized margins for placement about the legs of a wearer are disclosed in U.S. Pat. No. 4,050,462 and U.S. Pat. No. 5,092,861. An absorbent article having elasticized side margins and waist band margins are shown in U.S. Pat. No. 4,300,562.

Despite previous advancements in the field of absorbent articles, persons of ordinary skill in the art continue their efforts to produce absorbent articles having better absorbency and that are thus are better able to contain urinary and fecal excretions. For instance, problems with prior diaper designs include inferior absorbency and leakage of urinary or fecal material from the article. Prolonged contact of liquid or semi-solid excreta with the skin of the wearer is also a continuing problem in the art. For example, the moisture vapor and heat generated by the bodily exuded trapped within a diaper may lead conditions adjacent to wearer's skin which promotes skin irritation, infection, and the like. Various approaches to improve the absorbency of absorbent articles have been attempted, including the incorporation of a variety of absorbent composites into said absorbent articles.

Absorbent composites suitable for use in disposable absorbent garments such as diapers, adult incontinent products, feminine care products, training pants, and the like, are known. Generally, such absorbent composites comprise a means of containing a high-absorbency material and a high-absorbency material. Suitable means for containing the high-absorbency material include fibrous matrixes such as those formed from airlaid cellulose fibers or a coform material comprising cellulose fibers and meltblown polyolefin fibers. A wide variety of high-absorbency materials (also known as superabsorbent materials) are known to those skilled in the art.

For example, U.S. Pat. No. 5,843,059 to Niemeyer, et al. discloses an absorbent composite suitable for use in disposable absorbent garments and an absorbent garment including such a composite. The composite includes a means for containing a superabsorbent material and a superabsorbent material contained by the containment means. The reference specifies that the superabsorbent material has a Gel Integrity Index of at least about 1500 kg mm. Further, the superabsorbent material is present in the containment means in an amount of from about 10 to about 100 weight percent based on total weight of the containment means and the superabsorbent material.

Disposable absorbent garments formed from absorbent composites are intended to perform many uses. For example, disposable absorbent garments in the form of infant diapers are placed on children and are intended to absorb body fluids for a given period of time. During daytime use, care givers are generally readily available and will often change an infant's diaper after a single or perhaps two liquid insults. In contrast, the same diaper can be placed on an infant prior to the infant going to bed at night. This diaper may then stay on the infant until morning, a period of eight or more hours. This diaper will be subjected to three, four, or more liquid insults. Thus, a diaper intended for overnight use will have its absorbent ability taxed to a greater extent than a diaper intended for daytime use.

Similarly, cultural differences among different groups of people have been found to produce different diapering habits. That is, some cultural groups tend to change diapers more or less frequently than others. Thus, in certain cultures, even during daytime usage, a diaper may be subjected to three, four, or more liquid insults.

Unfortunately, diapers which perform completely satisfactory in circumstances where they are subjected to one or possibly two liquid insults can perform unsatisfactorily when subjected to three, four, or more liquid insults. This is highly undesirable.

It is possible to design diapers specific for daytime use and diapers specific for nighttime use. Unfortunately, this places a burden on the consumer to maintain proper supplies of both types of diapers. Moreover, it places the added burden on the consumer to time changing the diapers so that an overnight diaper is on the infant when the infant goes to sleep for the night. Such a solution to the described problem has been found generally unacceptable.

As is apparent from the foregoing, previous approaches present a variety of means for improving absorbency in absorbent garments. However, all of these proposed means are deficient in terms of effectiveness and low product quality, mechanical complexity in design, and/or associated cost inefficiencies.

In view of the deficiencies of the various products and processes disclosed in the above discussed references, it is highly desirable to provide cost-efficient absorbent articles that display superior absorbency, as well as novel compositions and composites for use in said absorbent articles. Further, it is desirable to provide an absorbent composite which is capable of exhibiting excellent leakage performance in both low loading usage situations and high loading usage situations. It is to this goal that the present invention is directed. Moreover, it is highly desirable to provide a cost-efficient process for producing absorbent articles having superior absorbency.

### SUMMARY OF THE INVENTION

The present invention provides absorbent articles, and novel compositions and composites for use in same, that display unexpectedly superior absorbency. Further, the present invention provides a cost-efficient process for producing absorbent articles having superior absorbency. Moreover, the present invention provides cost-efficient absorbent articles, methods for preparing and using such articles, and novel compositions and composites for use in same.

An embodiment of the present invention provides an absorbent article comprising: a substantially impermeable backsheet; a permeable topsheet; and an absorbent core disposed between the substantially impermeable backsheet and the permeable topsheet, said absorbent core comprising a superabsorbent polymer having a Gel Integrity Index (GII of less than about 500 kg mm.

A further embodiment of the present invention provides an absorbent article comprising: a substantially impermeable backsheet; a permeable topsheet; an absorbent core comprising about 30% to about 50% by weight of a superabsorbent polymer and about 50% to about 70% by weight of wettable fibers, said absorbent core being disposed between the substantially impermeable backsheet and the permeable topsheet, said superabsorbent polymer having a Gel Integrity Index (GII) of less than about 500 kg mm.

An even further embodiment of the present invention provides an absorbent article comprising: a substantially impermeable backsheet; a permeable topsheet; an absorbent core comprising about 30% to about 50% by weight of a crosslinked superabsorbent polymer, said absorbent core being disposed between the substantially impermeable backsheet and the permeable topsheet, said crosslinked superabsorbent polymer having a Gel Integrity Index (GII) of about 0.10 kg mm to about 0.30 kg mm and an AUL value of less than about 25 g/g.

A still further embodiment of the present invention provides an absorbent garment comprising: a substantially impermeable backsheet and a permeable topsheet defining a front waste portion and a rear waste portion, said front waste portion and said rear waste portion cooperating to form a waste opening; a crotch region formed between the front waste portion and the rear waste portion; a pair of leg openings on opposed sides of the crotch region; an absorbent core disposed between the substantially impermeable backsheet and the permeable topsheet at the crotch region; wherein the absorbent core comprises a superabsorbent polymer having a Gel Integrity Index (GII) of less than about 500 kg mm.

Another embodiment of the present invention provides a composition comprising: about 10% to about 80% by weight of a superabsorbent polymer, said superabsorbent polymer having a Gel Integrity Index (GII) of less than about 500 kg mm; and about 20% to about 90% by weight of wettable fibers.

Yet another embodiment of the present invention provides a composition prepared by a process comprising: combining about 10% to about 80% by weight of a superabsorbent polymer having a Gel Integrity Index (GII) of less than about 500 kg mm with about 20% to about 90% by weight of wettable fibers.

A further embodiment of the present invention provides a method of preparing a composition for use in absorbent articles comprising: combining wettable fibers with a superabsorbent polymer having a Gel Integrity Index of less than about 500 kg mm; wherein the wettable fibers comprise about 20% to about 90% by weight of the composition and the superabsorbent polymer comprises about 10% to about 80% by weight of the composition.

An even further embodiment of the present invention provides a method of preparing an absorbent article comprising: combining a superabsorbent polymer having a Gel Integrity Index of less than about 500 kg mm with wettable fibers to form an absorbent core and disposing the absorbent core between a substantially impermeable backsheet and a permeable topsheet.

### BRIEF DESCRIPTION OF THE DRAWINGS

FIG. 1A is perspective view, partially cutaway, of a disposable absorbent diaper according to an implementation of the present invention.

FIG. 1B is a vertical cross-sectional view of a section of the disposable absorbent diaper of FIG. 1A.

FIG. 2 illustrates the test probe used in the test method described herein for determining the Gel Integrity Index (GII).

FIG. 3 is a cutaway perspective view of a testing apparatus used to conduct the Fluid Intake Evaluation as described herein.

FIG. 4 is a side elevational view of the apparatus of FIG. 3.

### DETAILED DESCRIPTION OF THE INVENTION

As used herein, “absorbency” refers to the functional capacity and the rate at which absorption occurs as measured by absorption under load (AUL). “Air permeability”, as used herein, refers to the amount of air which the surface permits to pass through during a specified amount of time relative to another surface having the same total area as the first surface.

As used herein, the term “absorbent article” refers to articles that absorb and contain exudates, and more specifically refers to articles which are placed against or in proximity to the body of a wearer of the absorbent article to absorb and contain various exudates discharged from the body. A non-exhaustive list of examples of absorbent articles includes diapers, diaper cores, diaper covers, disposable diapers, training pants, feminine hygiene products and adult incontinence products, without limitation.

The term “disposable article” refers to absorbent articles that are intended to be discarded or partially discarded after a single use, i.e., they are not intended to be laundered or otherwise restored or reused. The term “unitary disposable absorbent article” refers to a disposable absorbent article that is essentially a single structure (i.e., it does not require separate manipulative parts such as a diaper cover and insert). As used herein, the term “diaper” refers to an absorbent article generally worn by infants and incontinent persons about the lower torso.

The claims are intended to cover all of the forgoing classes of absorbent articles, without limitation, whether disposable, unitary or otherwise. These classifications are used interchangeably throughout the specification, but are not intended to limit the claimed invention. The invention will be understood to encompass, without limitation, all classes of absorbent articles, including those described above. The absorbent article and the absorbent core therein may be formed in a variety of ways and the inventions is not intended to be limited to any specific manner of formation. Preferably, the absorbent core is thin in order to improve the comfort and appearance of a garment. The employance of thin, comfortable garments is disclosed, for example without limitation in U.S. Pat. No. 5,098,423 to Pineiak et al. which is herein incorporated by reference.

The present invention provides an absorbent article, as well as a method of preparing same and a method of using said absorbent article, having unexpectedly superior properties of absorbency, leakage protection and/or skin wellness. The present invention can be understood by the disclosure herein. The present invention is directed to absorbent articles, having unexpectedly superior absorbency properties. In accordance with an implementation of the present invention, absorbent articles comprise

The absorbent article in accordance with an implementation of the present invention comprises a superabsorbent polymer. Applicants have unexpectedly discovered that superabsorbent polymers having a Gel Integrity Index of less than about 500 kg mm provide superior absorbency characteristics. Any superabsorbent polymer having the physical characteristics recited herein are suitable. Persons of skill in the art would readily be able to selected and utilize such polymers to implement the present invention, based upon the guidance provided herein. Preferably, the superabsorbent polymer is a polyacrylate.

The preparation of such superabsorbent polymers and their incorporation into absorbent articles is described in further detail below and may be accomplished using conventional techniques and methods well known in the art. Persons of ordinary skill in the art would be readily able to prepare and identify superabsorbent polymers meeting these characteristics, without undue experimentation, based upon the guidance provided herein.

The present invention is unexpectedly effective with superabsorbent polymers having a low AUL. Preferably, the AUL is less than about 25 g/g at 0.3 psi. As used herein, AUL, refers to measurements at 0.3 psi of grams of a saline per grams polymer. The saline in the AUL measurements herein is a 0.9% by weight sodium chloride solution. The use of AUL as a measurement of absorbency is well known in the art. A person of ordinary skill in the art would readily understand how to use AUL as a measurement of absorbency, as described herein, in the selection of suitable superabsorbent polymers.

Optionally, the polymer is combined with a stabilizing agent. The stabilizing agent is preferably a crosslinking agent. Non-limiting exemplary stabilizing agents include formaldehyde, glutaraldehyde, glyoxal, glyoxylic acid, oxydisuccinic acid, citric acid, a dialdehyde having 2 to 8 carbon atoms, a monoaldehyde having an acid functionality and 2 to 8 carbon atoms, a polycarboxylic acid having 2 to 9 carbon atoms, and combinations thereof. The stabilizing agent is preferably selected from the group consisting of formaldehyde, glutaraldehyde, glyoxal, glyoxylic acid, oxydisuccinic acid, citric acid and combinations thereof. When the stabilizing agent is a crosslinking agent, the crosslinking agent may be selected from the group consisting of a dialdehyde having 2 to 8 carbon atoms, a monoaldehyde having an acid functionality and 2 to 8 carbon atoms, a polycarboxylic acid having 2 to 9 carbon atoms, and combinations thereof.

The absorbent core may additionally comprise a surfactant, a filler, an additive or a combination thereof. Preferably, the additive is selected from the group consisting of flame retardants, reinforcing agents, auxiliary blowing agents, medicaments, fragrances, colorants, cleaners, abrasives and combinations thereof.

The absorbent article may be, for example, a diaper, incontinent brief, training pant, diaper holder, diaper liner, sanitary napkin, hygienic garment or combinations thereof. Diapers may include daytime diapers, nighttime diapers, long-term wear diapers, travel diapers, swimming diapers, daytime/nighttime diapers, male diapers, female diapers, unisex diapers, active diapers, seasonal diapers, cold weather diapers, warm weather diapers, medicated diapers or combinations thereof.

The physical characteristics of the superabsorbent polymer in accordance with an implementation of the present invention are determined by various factors. These factors also influence the cost effectiveness of the absorbent articles. While a wide variety of superabsorbent materials are known, the present invention relates, in one aspect, to the proper selection of a superabsorbent material to allow formation of improved absorbent composites and disposable absorbent garments. U.S. Pat. No. 5,147,343 issued to Kellenberger, U.S. Pat. No. 4,673,402 issued to Weisman, U.S. Pat. No. 5,281,207 issued to Chmielewski et al., and U.S. Pat. No. 4,834,735 issued to Alemany, et al. disclose many types of polymers and methods for making them, and are incorporated herein by reference for all purposes and in a manner that is consistent herewith. Superabsorbent polymers and methods of making them are described in U.S. Pat. Nos. 4,666,983 and 4,734,478 issued to Tsubakimoto et al. which are incorporated herein by reference for all purposes and. in a manner that is consistent herewith. Also, U.S. Pat. No. 5,281,207 to Chmielewski, et al. generally discloses methods and materials for making an absorbent article and is also incorporated herein by reference for all purposes and in a manner that is consistent herewith.

Applicants have discovered that the performance of a superabsorbent material in absorbent composites intended for overnight usage or other usage situations, in which the absorbent composites will be subjected to high levels of liquid loading, depends, at least in part, on the gel characteristics of the superabsorbent material contained in the composite. As used herein, the gel characteristics of a superabsorbent material refers to the Gel Integrity Index herein described in greater detail in connection with the examples. Stated generally, the Gel Integrity Index is a measure of the resistance to flow as measured by penetration resistance of the gel slurry formed when the superabsorbent material is subjected to a high level of liquid loading.

Specifically, the Gel Integrity Index measures the resistance to penetration of the gel slurry formed by allowing a superabsorbent material to free swell in an aqueous solution containing 0.9 weight percent sodium chloride in which the superabsorbent material and sodium chloride solution are present in a weight ratio of 1:50. The Gel Integrity Index is measured as follows. First, mix one part by weight of superabsorbent material having a moisture content of less than 10 weight percent and 50 parts of an aqueous solution containing 0.9 weight percent sodium chloride, in a container having a 33 millimeter diameter and a height of 62 millimeters. Secondly, allow the superabsorbent material to swell for at least one hour until it appears to have generally reached equilibrium. Thirdly, measure the resistance of a sample of the mixture of swollen superabsorbent and aqueous solution of sodium chloride to penetration to a test probe, by attaching a clear anodized aluminum test probe having a 1.27 centimeter diameter, a length of 11.43 centimeters, and a rounded end having a 6.35 millimeter radius, to descend downward from a load cell capable of determining the load exerted on the load cell by the test probe; raising the sample of the mixture of swollen superabsorbent and aqueous solution of sodium chloride to the rounded end of the probe until a load greater than 0.1 gram but less than one gram is exerted on the load cell; lowering the test probe into the sample, for a distance of 40 millimeters at a constant speed of 16 inches per minute; and determining the resistance of the sample to the introduction of the test probe into the sample, as the probe penetrates from 15 to 40 millimeters into the sample. If the superabsorbent is in particulate form, the particles should preferably be screened to have a maximum cross-sectional diameter within the range of from about 50 microns to about 1000 microns, more preferably from about 100 microns to about 800 microns.

Superabsorbent materials suitable for use in the present invention have a Gel Integrity Index of at least about 500 kg mm. Preferably, the superabsorbent polymer has a Gel Integrity Index (GII) of less than about 10 kg mm. Even more preferably, the superabsorbent polymer has a Gel Integrity Index (GII) of less than about 1 kg mm. Even more preferably, the superabsorbent polymer has a Gel Integrity Index (GII) of less than about 0.05 kg mm. Most preferably, the superabsorbent polymer has a Gel Integrity Index (GII) of about 0.10 kg mm to about 0.30 kg mm. Applicants have discovered that absorbent cores comprising a superabsorbent polymer having a Gel Integrity Index as described above unexpectedly exhibit superior absorbency.

In addition to the superabsorbent materials described above, the absorbent composites according to the present invention comprise means to contain the superabsorbent material. Any means capable of containing the described superabsorbent materials, which means is further capable of being located in a disposable absorbent garment, is suitable for use in the present invention. Many such containment means are known to those skilled in the art. For example, the containment means may comprise a fibrous matrix such as an airlaid or wet-laid web of cellulosic fibers, a meltblown web of synthetic polymeric fibers, a spunbonded web of synthetic polymeric fibers, a coformed matrix comprising cellulosic fibers and fibers formed from the synthetic polymer material, airlaid, heat-fused webs of synthetic polymeric materials, open-celled foams, and the like.

Alternatively, the containment means may comprise two layers of material which are joined together to form a pocket or compartment, more particularly, a plurality of pockets, which pockets contain a superabsorbent material. In such a case, at least one of the layers of material should be water pervious. The second layer of material may be water pervious or water impervious. The layers of material may be clothlike woven or nonwovens, closed- or open-celled foams, perforated films, elastomeric materials, or may be fibrous webs of material. When the containment means comprises layers of material, the material should have a pore structure small enough or tortuous enough to contain the majority of superabsorbent material. The containment means may also comprise a laminate of two layers of material between which the superabsorbent material is located and contained.

Further, the absorbent article may comprise a fibrous matrix of wettable fibers wherein the superabsorbent polymer is disbursed. The superabsorbent polymer may be uniformly or unevenly disbursed. Moreover, the disbursement of the superabsorbent polymer may be targeted to attain optimal characteristics. The superabsorbent polymer may be non-affixed or affixed directly or indirectly to the fibrous matrix.

Any suitable amount of superabsorbent polymer may comprise the absorbent core. A person of skill in the art would readily be able to determine such a suitable amount without undue experimentation. Preferably, the superabsorbent polymer is about 10% to about 80% by weight of the absorbent core. More preferably, the superabsorbent polymer is about 20% to about 60% by weight of the absorbent core. Even more preferably, the superabsorbent polymer is about 30% to about 50% by weight of the absorbent core.

The absorbent core may further comprises wettable fibers. Any suitable amount of wettable fibers may comprise the absorbent core. A person of skill in the art would readily be able to determine such a suitable amount without undue experimentation. Preferably, the absorbent core additionally comprises about 20% to about 90% of wettable fibers. More preferably, the absorbent core additionally comprises about 50% to about 70% by weight of wettable fibers.

Any fibers capable of forming a containment means capable of containing a superabsorbent material and of forming a composite when in combination with the superabsorbent material are believed suitable for use in the present invention. It is often preferred that the fibers are hydrophilic. As used herein, a fiber will be considered to be “hydrophilic” when it possesses a contact angle of water in air of less than 90 degrees. For the purposes of this application, contact angle measurements are determined as set forth by Good and Stromberg in “Surface and Colloid Science,” Volume 11 (Plenum Press, 1979).

Fibers suitable for use in the present invention include cellulosic fibers such as wood pulp fluff, cotton, cotton linters, rayon, cellulose acetate, and the like, as well as synthetic polymeric fibers. The synthetic polymeric fibers may be formed from inherently hydrophilic polymeric materials or may be formed from inherently hydrophobic polymeric materials (water in air contact angle of greater than 90 degrees), which fibers are then treated to render at least the outer surface of the fibers hydrophilic. For example, hydrophilic fibers may be formed from an intrinsically hydrophilic polymer such as a block copolymer of nylon, e.g., nylon-6, and a polyethylene oxide diamine. Such block copolymers are commercially available from Allied Signal Inc. under the trade designation HYDROFIL™. Alternatively, the fibers may be formed from an intrinsically hydrophobic polymer such as polyolefin or polyester which has been surface modified to provide a generally nonfugitive hydrophilic surface.

When the hydrophilic fibers are formed by applying a hydrophilic surface treatment to a generally hydrophobic polymer, it is believed desirable to employ a generally nonfugitive surface treatment in order to obtain the desired performance standards. Absorbent structures employed in absorbent garments such as diapers are, as discussed above, often subjected to multiple liquid insults. If the surface treatment is fugitive, it may be washed off with the initial insult, thus, exposing the hydrophobic fiber surface. The hydrophobic fiber surface may impede the absorption performance of the absorbent structure. Of course, there are instances where hydrophobic fibers may be employed depending in part on the fluid to be absorbed.

The synthetic polymeric fibers suitable for use in the present invention may suitably be formed through a melt-extrusion process wherein fibers of a polymeric material are extruded and attenuated to produce fibers having a desired diameter. Alternatively the fibers may be formed through a spinning process. Any fiber-producing process known to those skilled in the art is believed to be suitable for use in the present invention.

Fibers suitable for use in the present invention generally have a length of at least about 1 millimeter. The fibers may have a maximum length approaching infinity. That is to say, the fibers may be essentially continuous, such as those fibers formed through a meltblowing process under certain conditions known to those skilled in the art.

Reference to a “mixture” is intended to refer to a combination of fibers and superabsorbent material in which the superabsorbent material is in direct contact with the fibers or is not substantially prevented from migrating into contact with the fibers. Thus, for example, in a multi-layered absorbent core in which the first layer comprises an airlaid mixture of wood pulp fluff and superabsorbent material and the second layer comprises only airlaid fluff, only the first layer is considered a “mixture” provided substantial dry migration of the superabsorbent material between the two layers is prevented. Methods of preventing such migration are known and include separating the layers by a tissue wrapsheet, high density fiber layer, or similar means to prevent substantial dry migration of the superabsorbent material between the two layers. The mixture of superabsorbent materials and fibers may be relatively homogenous or relatively nonhomogeneous. In the case of a nonhomogeneous mixture, the superabsorbent may be arranged in a gradient or may be layered with the fibers.

When the containment means comprises a mixture of fibers and a superabsorbent material, the mixture of fibers and the superabsorbent material may be formed in a wide variety of ways. For example, the mixture may be formed by air laying or wet laying the fibers in the superabsorbent material, according to processes known in the art, to form batts of the mixture. Air laying the mixture of fibers and superabsorbent material is intended to encompass both the situation wherein preformed fibers are air laid with the superabsorbent material as well as the situation in which the superabsorbent material is mixed with the fibers as the fibers are being formed, such as through a meltblowing process.

The absorbent composites according to the present invention are suited to absorb many fluids, including body fluids such as urine, menses, and blood; and are suited for use in absorbent garments such as diapers, adult incontinent products, bed pads, and the like; in catamenial devices such as sanitary napkins, tampons, and the like; and in other absorbent products such as wipes, bibs, wound dressings, food packaging and the like. Accordingly, in another aspect, the present invention relates to a disposable absorbent garment comprising an absorbent composite as described above. A wide variety of absorbent garments are known to those skilled in the art. The absorbent composites of the present invention can be incorporated into such known absorbent garments. Exemplary absorbent garments are generally described in U.S. Pat. Nos. 4,710,187 issued Dec. 1, 1987, to Boland et al.; 4,762,521 issued Aug. 9, 1988, to Roessler et al.; 4,770,656 issued Sep. 13, 1988, to Proxmire et al.; 4,798,603 issued Jan. 17, 1989, to Meyer et al.; 5,411,497 issued May 2, 1995 to Tanzer et al.; 5,433,715 issued Jul. 18, 1995 to Tanzer et al.; 5,425,725 issued Jun. 20, 1995 to Tanzer et al.; and commonly assigned U.S. patent application Ser. No. 08/096,654, now U.S. Pat. No. 5,509,915 filed Jul. 22, 1993, as a continuation of Ser. No. 07/757,760, filed Sep. 11, 1991, and now abandoned in the name of Hanson et al. (EP 0 539 703) and Ser. No. 08/369,558 now U.S. Pat. No. 5,593,399 filed Jan. 6, 1995, as a continuation of Ser. No. 145,926, filed Oct. 29, 1993 in the name of Tanzer et al., all of which are incorporated herein by reference. As a general rule, the absorbent disposable garments according to the present invention comprise a bodyside liner adapted to contact the skin of a wearer, an outer cover superimposed in facing relation with said liner, and an absorbent composite, such as those described above, superimposed on said outer cover and located between the bodyside liner and the outer cover. Those skilled in the art will recognize materials suitable for use as the bodyside liner and outer cover. Examples of materials suitable for use as the bodyside liner are hydrophilized spunbond polypropylene or polyethylene with a basis weight of from about 15 to about 25 grams per square meter, and the like. Examples of materials suitable for use as the outer cover are water-impervious materials such as polyolefin films, as well as water-pervious or water vapor-pervious materials.

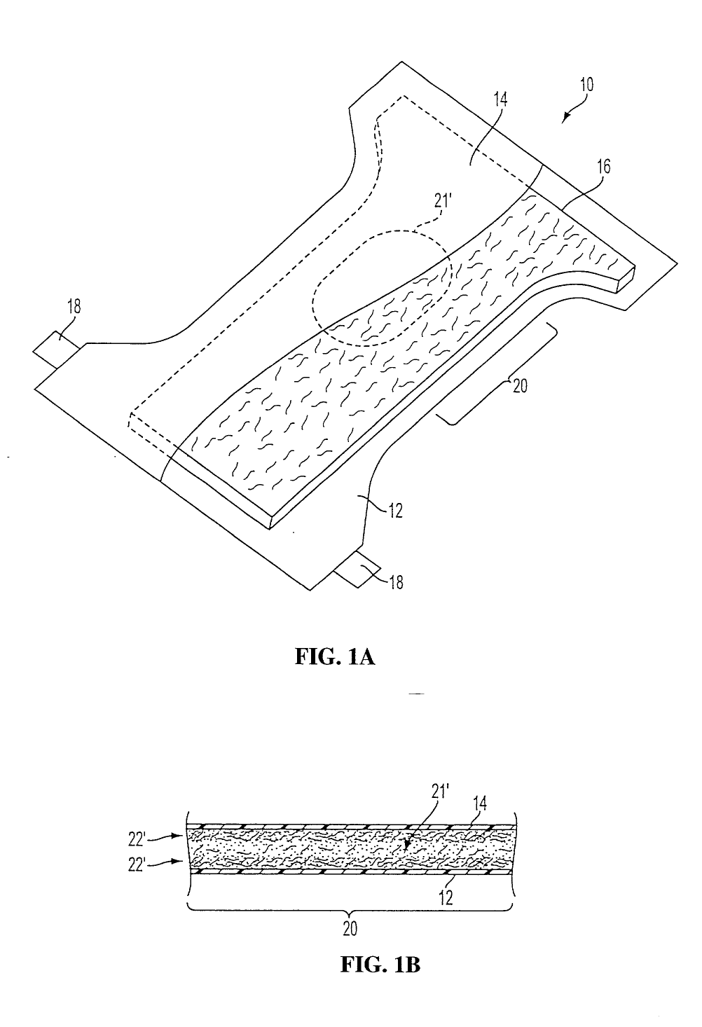

The present invention also provides improved disposable absorbent articles such as but not limited to diapers, sanitary napkins that incorporate the absorbent composite of the present invention. Disposable diaper articles are described in U.S. Pat. Nos. 4,673,402; 5,147,343; 5,330,822; 4,834,735; and 5,281,207, which are incorporated herein by reference for all purposes. A preferred disposable diaper, for the purpose of this invention, is shown in FIGS. 1A and 1B. In accordance with FIGS. 1A and 1B, a disposable diaper 10 comprises a liquid impermeable backsheet 12, a liquid permeable topsheet 14 and an absorbent core 16 positioned between the topsheet 14 and the backsheet 12.

In accordance with an implementation of the present invention, in at least a layer of the absorbent core, in a target region thereof indicated by circle 21′, taken in the Z-direction thereof (i.e., in a direction from top to bottom, away from the wearer), the superabsorbent material comprises a substantially continuous phase. For purposes of this disclosure, the substantially continuous phase is provided wherein a sufficient quantity of particles of the superabsorbent material are in multiple point contact with each other, both prior to absorption of liquid and thereafter, to thereby define a capillary network for facilitating liquid transport within the core structure. A sufficiently quantity of wood pulp fibers are intermixed with the superabsorbent material in the continuous phase. This quantity of wood pulp fiber acts to maintain the stability of the absorbent structure by integrating the region of the continuous phase of superabsorbent particles with adjacent portions of the absorbent structure.

As shown in the cross-sectional view FIG. 1B, the layer of the absorbent core having the continuous phase portion 21′ is preferably positioned between two layers, designated 22′, each comprising predominantly wood pulp fibers. In the case of a diaper, the liquid permeable top sheet 14 allows urine to flow through the sheet to the absorbent core 16 and also keeps the baby from directly contacting the absorbent panel structure. This configuration provides more comfort for the baby and also helps to position the absorbent panel structure. Liquid permeable top sheets, and liquid impermeable back sheets, are well known to those skilled in the art, and these components can be suitably selected in practicing the present invention.

Backsheet 12 is impermeable to liquids, and thus, helps to retain a liquid so that the liquid may be absorbed and retained by the absorbent panel structure. In a baby diaper, the impermeable back sheet is typically a sheet of plastic film, such as polyethylene, that helps to retain the urine so that the urine may be absorbed by the absorbent panel structure of the diaper. For a detailed discussion of materials that can be used in the top and back sheet of a diaper, see U.S. Pat. No. 5,281,207 issued to Chmielewski, et al. and which is incorporated herein for all purposes.

The absorbent core 16 is optionally made of a two phase matrix comprising wood pulp fiber and surface crosslinked polymeric superabsorbent material. As noted above, by two phase, it is meant that the absorbent core has two components, fibers (preferably wood pulp) and a superabsorbent material. The absorbent structure may comprise more than one layer. For example, the absorbent structure may have a layer that is substantially wood pulp fiber, while on top of this layer the absorbent structure may have another layer of wood pulp fiber that contains particulate superabsorbent material dispersed in the wood pulp fiber. It is contemplated that many different combinations of layers may be used in the practice of the present invention. For example, in a preferred embodiment of the invention, in at least the target region 20, a three layer system is formed in which a layer containing superabsorbent particulate material in a substantially continuous phase is positioned between adjacent layers formed predominantly of wood pulp fiber.

Because superabsorbent material is one of the most costly components of an absorbent structure, efficient use and positioning of the material is beneficial. Specific positioning of the superabsorbent material in areas most likely to be insulted with urine allows for the most cost effective utilization of this component. Specific positioning of superabsorbent material can be accomplished through any of several methods, such as by the method and apparatus as described and claimed in U.S. Pat. No. 5,279,854, which is incorporated herein by reference.

Persons of ordinary skill in the art would readily be able to use a variety of conventional techniques to produce superabsorbent polymers having the Gel Integrity Index characteristics disclosed herein, in accordance with an implementation of the present invention. Persons of ordinary skill in the art would be readily able to vary other characteristics discussed herein to obtain the superabsorbent polymers in accordance with the present invention, using conventional materials and techniques. Further, various techniques and methods for measuring such characteristics are well known in the art.

Absorbent cores having suitable superabsorbent polymeric composition and the structural features hereinbefore described will, in general, possess mechanical properties, e.g., resistance to compression deflection, flexibility, recovery from compression deflection, integrity, softness, etc., which render such materials suitable for use as absorbent structures in absorbent articles such as disposable diapers. Within the aforementioned structural limitations, however, it is possible to select certain combinations of parameters and/or certain preparation techniques and conditions which provide absorbents that exhibit especially desirable mechanical properties.

Thus, the superabsorbent polymer of the preferred structures herein will possess both viscous, i.e., fluid-like, properties and elastic, i.e., spring-like, properties. It is important that the superabsorbent polymeric material which forms the structure have physical, Theological, and morphological attributes which, under conditions of use, impart suitable flexibility, resistance to compression deflection, and dimensional stability to the absorbent composition material.

The superabsorbent polymers of the present invention can be prepared using any suitable polymerization and post-polymerization process steps and using any suitable combination of monomeric materials, so long as superabsorbent polymers result which have the hereinbefore described essential, and if desired preferred, structural and mechanical characteristics.

The absorbent core of the absorbent article embodiments of this invention can consist solely of one or more of the composition structures herein. For example, the absorbent core may comprise a single unitary piece of composition shaped as desired or needed to best fit the type of absorbent article in which it is to be used. Alternatively, the absorbent core may comprise a plurality of composition pieces or particles which may be adhesively bonded together or which may simply be constrained into an unbonded aggregate held together by an overwrapping of envelope tissue or by means of the topsheet and backing sheet of the absorbent article.

The absorbent core of the absorbent articles herein can also comprise other, e.g., conventional, elements or materials in addition to one or more absorbent structures of the present invention. For example, absorbent articles herein may utilize an absorbent core which comprises a combination, e.g., an airlaid mixture, of particles or pieces of the absorbent structures herein and conventional absorbent materials such as a) wood pulp or other cellulosic fibers, and/or, b) particles or fibers of polymeric gelling agents.

In one embodiment involving a combination of the absorbent material herein and other absorbent materials, the absorbent articles herein may employ a multi-layer absorbent core configuration wherein a core layer containing one or more structures of this invention may be used in combination with one or more additional separate core layers comprising conventional absorbent structures or materials. Such conventional absorbent structures or materials, for example, can include air-laid or wet-laid webs of wood pulp or other cellulosic fibers. Such conventional structures may also comprise conventional absorbent compositions.

As indicated hereinbefore, the fluid handling and mechanical characteristics of the specific absorbent structures herein render such structures especially suitable for use in absorbent articles in the form of disposable diapers. Disposable diapers comprising the absorbent structures of the present invention may be made by using conventional diaper making techniques, but by replacing or supplementing the pulp fibers or modified cellulosic core absorbents typically used in conventional diapers with one or more structures of the present invention. Absorbent structures of this invention may thus be used in diapers in single layer or, as noted hereinbefore, in various multiple layer core configurations.

Another preferred type of absorbent article which can utilize the absorbent structures of the present invention comprises form-fitting products such as training pants. Such form-fitting articles will generally include a nonwoven, flexible substrate fashioned into a chassis in the form of briefs or shorts. An absorbent structure according to the present invention can then be affixed in the crotch area of such a chassis in order to serve as an absorbent “core”. This absorbent core will frequently be over-wrapped with envelope tissue or other liquid pervious, nonwoven material. Such core overwrapping thus serves as the topsheet for the form-fitting absorbent article.

Due to the wide variety of materials which may be incorporated into the absorbent articles of the present invention, the present invention is not intended to be limited to any specific materials. The topsheet, backsheet, absorbent core and other components of the absorbent articles in accordance with various implementations of the present invention may comprise various materials. Persons of ordinary skill in the art would be readily able to select appropriate materials for use in the various components of the present invention based upon the materials.

In accordance with various implementations of the present invention, the absorbent core may contain one or more wettable fibers, one or more polymers or combinations thereof. Non-limiting exemplary fibers which may be used in the articles of the present invention include, without limitation, cellulose fibers, cellulose acetate fibers, rayon fibers, Courtauld's LYOCEL fibers, polyacrylonitrile fibers, surface modified (hydrophilic) polyester fibers, surface modified polyolophin/polyester bicomponent fibers, surface modified polyester/polyester bicomponent fibers, cotton fibers or blends thereof. Preferably cellulose acetate, rayon, Courtauld's LYOCEL, polyacrylonitrile, cotton fibers and cotton linters or combinations thereof are used in the process of the present invention. More preferably, cellulose fibers are used as the fiber material in the present invention.

Other materials may be added to the fiber or pulp material which is optionally processed in a fiberizing apparatus, such as a hammermill. The additives may be added at any point in the process. Non-limiting exemplary additives which may be incorporated into the process of the present invention include a polymer such as a superabsorbent polymer, hydrophilic polymers, potato starch, corn starch, wheat starch or rice starch, or combinations thereof. Various different combinations of materials may be used as are known to persons of ordinary skill in the art and which are described in U.S. Pat. No. 6,068,620 which is herein incorporated by reference. Preferably, the mixtures incorporated in the invention are substantially homogenous mixtures or uniformly distributed mixtures. Absorbent articles in accordance with an implementation of the present invention are prepared using conventional methods and materials well known to persons of ordinary skill in the art, using the guidelines provided herein.

The test method for determining the Gel Integrity Index of the superabsorbent polymer in accordance with the present invention is provided in U.S. Pat. No. 5,843,050, issued on Dec. 1, 1998 to Niemeyer et al., which is incorporated herein by reference in its entirety.

### TEST METHODS

Moisture Content of Superabsorbent Material

The following test method is suitably used to determine the moisture content of superabsorbent material.

Equipment Used:

1. An electronic balance accurate to 0.001 gram, such as that available from Sartorius Co. under the trade designation BP310S.

2. A forced-air oven capable of maintaining an internal temperature of 105 degrees C. plus or minus 2 degrees C. Such an oven is commercially available from Blue M under the designation Stabil-Therm.

3. A desiccator containing fresh calcium chloride. Such a desiccator can be obtained from Baxter Scientific Co. under the designation Pyrex Knob Top Desiccator.

4. A 60 millimeter aluminum weighing dish, such as that available from Sargent Welch Co. under the designation #S 25725.

Test Procedure:

1. Preheat oven to 105 degrees C. plus or minus 2 degrees C.

2. Weigh the aluminum weighing dish and record the weight as W1.

3. Place 8-10 grams of superabsorbent material in the weighing dish.

4. Weigh the weighing dish and superabsorbent material and record the weight as W2.

5. Place the weighing dish and superabsorbent material in the preheated oven for 3 hours.

6. Remove the weighing dish and superabsorbent material from the oven and place in desiccator. Allow to cool for approximately 30 minutes.

7. Remove the weighing dish and superabsorbent material from the desiccator and immediately weigh the cooled weighing dish and superabsorbent material. Record the weight as W3.

8. The percent moisture is calculated by the formula:

% moisture=100×(W2−W3)/(W2−W1)

Gel Integrity Index:

The following test method is used for determining the Gel Integrity Index (GII) of superabsorbent polymers in accordance with the present invention.

Equipment Used

1. An electronic balance accurate to 0.001 gram, such as that available from Sartorius Co. under the trade designation BP310S.

2. U.S. Standard 30 mesh and 50 mesh screens, automatic sieve shaker, such as a Ro-Tap Sieve Shaker commercially available from Baxter Scientific.

3. Air tight glass containers, such as those available from Baxter Scientific under the trade designation Qorpak Bottles AP-2103.

4. 0.87 percent aqueous saline solution commercially available from Baxter Scientific under the trade designation Blood Bank Saline.

5. A 33 millimeter diameter by 62 millimeter high, 55 milliliter capacity polystyrene vial, such as that commercially available from Baxter Scientific under the trade designation Continental Glass and Plastic Co. Polystyrene Snap Cap Vial.

6. A Tensile Tester, such as that commercially available from Instron under the trade designation Model #1 122. The Tensile Tester is interfaced with a personal computer including Windows.®. and Test Works.®. for Windows software.

7. A 2000 gram compression load cell for the Tensile Tester of No. 6, which load cell is commercially available from Instron.

8. Test Works.™. software commercially available from Sintech under the trade designation Test Works for Windows.

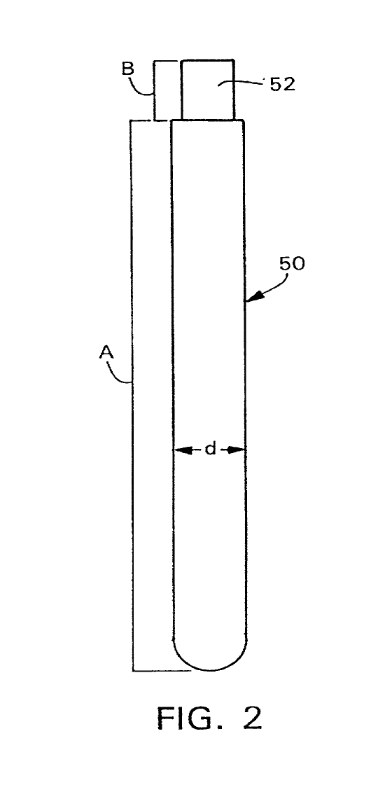

9. A 1.27 centimeter diameter (d) clear anodized aluminum test probe as illustrated in FIG. 2. With reference to FIG. 2, the test probe 50 has a length A of 11.43 centimeters. The test probe 50 has a threaded portion 52 having a length B of 1 centimeter. The threaded portion 52 is adapted to screw into the load cell of number 7 above. The end of probe 50 opposite threaded portion 52 is rounded (0.635 centimeter radius).

10. A laboratory jack

11. Polystyrene weigh boat, commercially available from Baxter Scientific under the trade designation S/P Brand dispo Weigh Boat Containers.

Sample Preparation:

1. Take a quantity of superabsorbent material as it is received from the superabsorbent supplier, but having a moisture content of less than 10 weight percent, and prescreen according to ASTM Test Method D-1921. If the superabsorbent material has a moisture content of greater than 10 weight percent, it should be dried at about 105 degrees C. until it has a moisture content of less than 10 weight percent. Fibrous superabsorbent materials do not need to be prescreened but should have (or be dried to) a moisture content of less than 10 weight percent. Transfer the superabsorbent material passing through the U.S. Standard 30 mesh screen and retained on the U.S. Standard 50 mesh screen (300-600 micron portion) of the superabsorbent material into the air tight container to prevent moisture pick-up.

2. Transfer 40 milliliters (+0.01 milliliter) of the 0.87 percent Baxter Blood Bank saline into the polystyrene vial.

3. Measure 0.80 gram of the superabsorbent material obtained under No. 1 above (300-600 micron particle size) into the polystyrene weigh boat. Transfer the superabsorbent material from the weigh boat into the polystyrene vial, place the cap on the vial, and swirl gently for 10 seconds. After swirling, allow the superabsorbent material to swell undisturbed at room temperature for one to eight hours. The superabsorbent is allowed to swell until it appears to have generally reached equilibrium (has stopped swelling). One hour is generally sufficient to reach this apparent equilibrium stage. Transfer the vial with as little movement as possible to the platform of the tensile tester. Triplicate samples for each superabsorbent material to be tested are prepared.

Test Set-Up:

1. Plug the 2000 gram compression load cell into the tensile tester and allow it to warm up for at least 30 minutes.

2. Turn on the personal computer and enter the Windows.™. program.

3. Enter the Testworks™ software.

4. Set the parameters in the compression master preset program as provided in U.S. Pat. No. 5,843,059 incorporated herein:

5. Calibrate the load cell to within 1 percent according to the calibration procedure supplied with the Tensile Tester.

6. Once calibrated, mount the load cell to the Tensile Tester frame and attach the 1.27 centimeter diameter test probe to the load cell.

Test Procedure:

1. Click on “sample” at the top of the screen and type in a sample description. Press enter.

2. Zero the load on the load cell.

3. Remove the cap from one of the polystyrene vials containing the swollen superabsorbent material prepared as described above and place it on a laboratory jack located below the test probe which has been securely screwed into the load cell.

4. Raise the sample with the laboratory jack until a load greater than 0.1 g but less than 1 g is exerted on the load cell.

5. Re-zero the load on the load cell and then click on “run” at the top of the screen.

6. The Instron machine will then lower the 1.27 centimeter diameter test probe into the sample for a distance of 40 mm at a constant speed of 16 inches per minute.

7. When the probe stops, press “return” on the Instron panel and raise the probe from the sample. Wipe off the probe. Test the remaining two samples of the same material in the manner described above.

Print the results of the testing. The Gel Integrity Index is the area under the curve generated by the Tensile Tester equipment from initiation of the test procedure to the end of the procedure for the three test samples. The value is reported as the average energy of the three replications in kg mm.

Fluid Intake Evaluation:

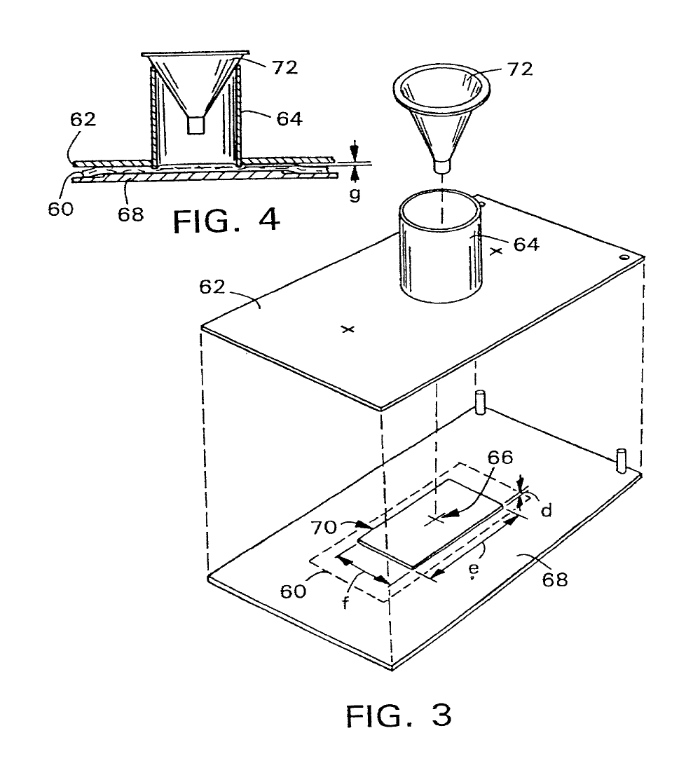

FIGS. 3 and 4 illustrate the test apparatus used to conduct the fluid intake evaluation. With reference to FIG. 3, a 4 inch×10 inch test sample (shown in phantom at 60) is provided. The test sample 60 is placed flat and smooth under a cylinder plate assembly 62 such that the cylinder 64, which has a 5.1 centimeter internal diameter, is positioned over the center 66 of lower plate 68 and raised platform 70. Plate 62 and 68 are 14 inches long and 8 inches wide and are formed from a material such as Plexiglas. Raised platform 70 is ½ inch high (d) by 6 inches long (e) by 3 inches wide (f). The cylinder 64 extends a distance (g) of about 1/32 inch below the cylinder plate assembly 62. This can be seen by reference to FIG. 4. Funnel 72 is sized to fit in cylinder 64 and has a receiving end which is 7 centimeters in diameter and an exit end which is 1.2 centimeters in diameter.

The fluid intake evaluation is performed as follows. The 4 inch by 10 inch test sample 60 is positioned over raised platform 70 such that it is centered thereon. Two 3 inch by 11 inch strips of blotter paper are provided. The blotter paper is 100 pound blotter paper commercially available from James River Corporation under the trade designation 100 pound Verigood Blotter Paper. Each blotter paper strip is weighed and its weight recorded. One strip of blotter paper is placed immediately adjacent, but not touching, each longtudinal side (10 inch) of the test sample 60. Cylinder plate assembly 62 is placed on top of lower plate 68 such that they are superimposed on one another. Two cylindrical weights are placed at the areas marked with an “x” (FIG. 3) so that a 0.836 pound per square inch weight is applied to the 3″.times.6″ portion of test sample 60 located on the raised platform 70, (less the area under cylinder 64). The total weight applied is approximately 12.4 pounds (5623 grams).

Sixty milliliters of a 0.87 percent aqueous saline solution commercially available from Baxter Scientific under the trade designation Blood Bank Saline is poured through funnel 72 in cylinder 64 so as to reach test sample 60. The time required for the 60 milliliter saline solution to disappear from the surface of the test sample 60 is recorded. After the fluid disappears from the surface of the test sample, the blotter strips are removed and weighed to determine the amount of liquid absorbed by the blotter strips. New blotter strips are weighed and placed next to the test sample and a second 60 milliliter insult is applied through the funnel 10 minutes after the first insult was applied. Again, the time required for the fluid to disappear from the surface of the test sample is recorded. The blotter strips are removed and weighed to determine the amount of liquid absorbed by the blotter strips. The procedure of replacing the blotter strips with new strips, insulting the test sample with 60 milliliters of Blood Bank Saline, determining the amount of time necessary for the fluid to disappear from the surface of the test sample 60 and determining the amount of liquid absorbed by the blotter strips is repeated for a total of 5 insults. Each insult occurs 10 minutes after the prior insult. The amount of leakage for each insult is determined by subtracting the dry blotter weights from the wet blotter weights for the blotter strips used for that insult. The intake rate for each insult is determined by subtracting the leakage from the 60 milliliter insult and dividing by the intake time>(60 ml-leakage)/intake time (min)

### EXAMPLES

### Example 1

Comparative Study of Absorbent Articles

A comparative study was conducted on the absorbency of absorbent articles in accordance with an implementation of the present invention (Sample 1) and a representative conventional diaper (Pampers Baby Dry). The results of the study are displayed in Table 1 below:

TABLE 1

| | | | | | | | |
|---|---|---|---|---|---|---|---|
| TESTED DIAPER | PRESSURE | 0.1 | 0.1 | 0.1 | 0.5 | 0.5 | 0.5 |
| | (PSI) | | | | | | |
| | DOSE | 1 | 2 | 3 | 1 | 2 | 3 |
| Sample 1 | Absorption | 2 | 2 | 2 | 2 | 3 | 5 |
| | Times (s) | | | | | | |
| Pampers Baby Dry | Absorption | 4 | 4 | 9 | 5 | 10 | >600 |
| | Times (s) | | | | | | |

The present invention has been described in connection with the preferred embodiments. These embodiments, however, are merely for example and the invention is not restricted thereto. Any examples described herein are illustrative of preferred embodiments of the inventive subject matter and are not to be construed as limiting the inventive subject matter thereto. It will be understood by those skilled in the art that other variations and modifications can easily be made within the scope of the invention as defined by the appended claims.

What is claimed is:

1. An absorbent article comprising:
a substantially impermeable backsheet;
a permeable topsheet; and
an absorbent core disposed between the substantially impermeable backsheet and the permeable topsheet, said absorbent core comprising a superabsorbent polymer having a Gel Integrity Index (GII) of less than about 500 kg mm.

2. The absorbent article of claim 1, wherein the superabsorbent polymer is about 10% to about 80% by weight of the absorbent core.

3. The absorbent article of claim 1, wherein the superabsorbent polymer is about 20% to about 60% by weight of the absorbent core.

4. The absorbent article of claim 1, wherein the superabsorbent polymer is about 30% to about 50% by weight of the absorbent core.

5. The absorbent article of claim 1, wherein the absorbent core additionally comprises about 50% to about 70% by weight of wettable fibers.

6. The absorbent article of claim 1, wherein the superabsorbent polymer has an AUL value of less than about 25 g/g.

7. The absorbent article of claim 1, wherein the superabsorbent polymer is crosslinked.

8. The absorbent article of claim 1, wherein the superabsorbent polymer is a polyacrylate.

9. The absorbent article of claim 1, wherein the superabsorbent polymer has a Gel Integrity Index (GII) of less than about 10 kg mm.

10. The absorbent article of claim 1, wherein the superabsorbent polymer has a Gel Integrity Index (GII) of less than about 1 kg mm.

11. The absorbent article of claim 1, wherein the superabsorbent polymer has a Gel Integrity Index (GII) of less than about 0.05 kg mm.

12. The absorbent article of claim 1, wherein the superabsorbent polymer has a Gel Integrity Index (GII) of about 0.10 kg mm to about 0.30 kg mm.

13. The absorbent article of claim 1, wherein the absorbent core additionally comprises a surfactant, a filler, an additive or a combination thereof.

14. The absorbent article of claim 13, wherein the additive is selected from the group consisting of a flame retardant, a reinforcing agent, an auxiliary blowing agent, a medicament, a fragrance, a colorant, a cleaner, an abrasive and a combination thereof.

15. The absorbent article of claim 1, wherein the absorbent article is a diaper, incontinent brief, training pant, diaper holder, diaper liner, sanitary napkin, hygienic garment or combination thereof.

16. An absorbent article comprising:
a substantially impermeable backsheet;
a permeable topsheet;
an absorbent core comprising about 30% to about 50% by weight of a superabsorbent polymer and about 50% to about 70% by weight of wettable fibers, said absorbent core being disposed between the substantially impermeable backsheet and the permeable topsheet, said superabsorbent polymer having a Gel Integrity Index (GII) of less than about 500 kg mm.

17. The absorbent article of claim 16, wherein the superabsorbent polymer has an AUL value of less than about 25 g/g.

18. The absorbent article of claim 16, wherein the superabsorbent polymer is crosslinked.

19. The absorbent article of claim 16, wherein the superabsorbent polymer is a polyacrylate.

20. The absorbent article of claim 16, wherein the superabsorbent polymer has a Gel Integrity Index (GII) of less than about 10 kg mm.

21. The absorbent article of claim 16, wherein the superabsorbent polymer has a Gel Integrity Index (GII) of less than about 1 kg mm.

22. The absorbent article of claim 16, wherein the superabsorbent polymer has a Gel Integrity Index (GII) of less than about 0.05 kg mm.

23. The absorbent article of claim 16, wherein the superabsorbent polymer has a Gel Integrity Index (GII) of about 0.10 kg mm to about 0.30 kg mm.

24. The absorbent article of claim 16, wherein the absorbent core additionally comprises a surfactants, a filler, an additive or a combination thereof.

25. The absorbent article of claim 24, wherein the additive is selected from the group consisting of a flame retardant, a reinforcing agent, an auxiliary blowing agent, a medicament, a fragrance, a colorant, a cleaner, an abrasive and a combination thereof.

26. The absorbent article of claim 16, wherein the absorbent article is a diaper, incontinent brief, training pant, diaper holder, diaper liner, sanitary napkin, hygienic garment or combination thereof.

27. An absorbent article comprising:
a substantially impermeable backsheet;
a permeable topsheet;
an absorbent core comprising about 30% to about 50% by weight of a crosslinked superabsorbent polymer, said absorbent core being disposed between the substantially impermeable backsheet and the permeable topsheet, said crosslinked superabsorbent polymer having a Gel Integrity Index (GII) of about 0.10 kg mm to about 0.30 kg mm and an AUL value of less than about 25 g/g.

28. An absorbent garment comprising:
a substantially impermeable backsheet and a permeable topsheet defining a front waste portion and a rear waste portion, said front waste portion and said rear waste portion cooperating to form a waste opening;
a crotch region formed between the front waste portion and the rear waste portion;
a pair of leg openings on opposed sides of the crotch region;
an absorbent core disposed between the substantially impermeable backsheet and the permeable topsheet at the crotch region;
wherein the absorbent core comprises a superabsorbent polymer having a Gel Integrity Index (GII) of less than about 500 kg mm.

29. The absorbent garment of claim 28, wherein the superabsorbent polymer is about 10% to about 80% by weight of the absorbent core.

30. The absorbent garment of claim 28, wherein the superabsorbent polymer is about 20% to about 60% by weight of the absorbent core.

31. The absorbent garment of claim 28, wherein the superabsorbent polymer is about 30% to about 50% by weight of the absorbent core.

32. The absorbent garment of claim 28, wherein the absorbent core additionally comprises about 50% to about 70% by weight of wettable fibers.

33. The absorbent garment of claim 28, wherein the superabsorbent polymer has an AUL value of less than about 25 g/g.

34. The absorbent garment of claim 28, wherein the superabsorbent polymer is crosslinked.

35. The absorbent garment of claim 28, wherein the superabsorbent polymer is a polyacrylate.

36. The absorbent garment of claim 28, wherein the superabsorbent polymer has a Gel Integrity Index (GII) of less than about 10 kg mm.

37. The absorbent garment of claim 28, wherein the superabsorbent polymer has a Gel Integrity Index (GII) of less than about 1 kg mm.

38. The absorbent garment of claim 28, wherein the superabsorbent polymer has a Gel Integrity Index (GII) of less than about 0.05 kg mm.

39. The absorbent garment of claim 28, wherein the superabsorbent polymer has a Gel Integrity Index (GII) of about 0.10 kg mm t o about 0.30 kg mm.

40. The absorbent garment of claim 28, wherein the absorbent core additionally comprises a surfactant, a filler, an additive or a combination thereof.

41. The absorbent garment of claim 40, wherein the additive is selected from the group consisting of a flame retardant, a reinforcing agent, an auxiliary blowing agent, a medicament, a fragrance, a colorant, a cleaner, an abrasive and a combination thereof.

42. A composition comprising:
about 10% to about 80% by weight of a superabsorbent polymer, said superabsorbent polymer having a Gel Integrity Index (GII) of less than about 500 kg mm; and
about 20% to about 90% by weight of wettable fibers.

43. The composition of claim 42, wherein the superabsorbent polymer is about 20% to about 60% by weight of the composition.

44. The composition of claim 42, wherein the superabsorbent polymer is about 30% to about 50% by weight of the composition.

45. The composition of claim 42, wherein the superabsorbent polymer has an AUL value of less than about 25 g/g.

46. The composition of claim 42, wherein the superabsorbent polymer is crosslinked.

47. The composition of claim 42, wherein the superabsorbent polymer is a polyacrylate.

48. The composition of claim 42, wherein the superabsorbent polymer has a Gel Integrity Index (GII) of less than about 10 kg mm.

49. The composition of claim 42, wherein the superabsorbent polymer has a Gel Integrity Index (GII) of less than about 1 kg mm.

50. The composition of claim 42, wherein the superabsorbent polymer has a Gel Integrity Index (GII) of less than about 0.05 kg mm.

51. The composition of claim 42, wherein the superabsorbent polymer has a Gel Integrity Index (GII) of about 0.10 kg mm to about 0.30 kg mm.

52. A composition prepared by a process comprising:
combining about 10% to about 80% by weight of a superabsorbent polymer having a Gel Integrity Index (GII) of less than about 500 kg mm with about 20% to about 90% by weight of wettable fibers.

53. The composition of claim 52, wherein the superabsorbent polymer is about 20% to about 60% by weight of the composition.

54. The composition of claim 52, wherein the superabsorbent polymer is about 30% to about 50% by weight of the composition.

55. The composition of claim 52, wherein the superabsorbent polymer has an AUL value of less than about 25 g/g.

56. The composition of claim 52, wherein the superabsorbent polymer is crosslinked.

57. The composition of claim 52, wherein the superabsorbent polymer is a polyacrylate.

58. The composition of claim 52, wherein the superabsorbent polymer has a Gel Integrity Index (GII) of less than about 10 kg mm.

59. The composition of claim 52, wherein the superabsorbent polymer has a Gel Integrity Index (GII) of less than about 1 kg mm.

60. The composition of claim 52, wherein the superabsorbent polymer has a Gel Integrity Index (GII) of less than about 0.05 kg mm.

61. The composition of claim 52, wherein the superabsorbent polymer has a Gel Integrity Index (GII) of about 0.10 kg mm to about 0.30 kg mm.

62. A method of preparing a composition for use in absorbent articles comprising:
combining wettable fibers with a superabsorbent polymer having a Gel Integrity Index of less than about 500 kg mm;
wherein the wettable fibers comprise about 20% to about 90% by weight of the composition and the superabsorbent polymer comprises about 10% to about 80% by weight of the composition.

63. The method of claim 62, wherein the superabsorbent polymer is about 20% to about 60% by weight of the composition.

64. The method of claim 62, wherein the superabsorbent polymer is about 30% to about 50% by weight of the composition.

65. The method of claim 62, wherein the wettable fibers comprises about 50% to about 70% by weight of the composition.

66. The method of claim 62, wherein the superabsorbent polymer has an AUL value of less than about 25 g/g.

67. The method of claim 62, wherein the superabsorbent polymer has a Gel Integrity Index (GII) of less than about 10 kg mm.

68. The method of claim 62, wherein the superabsorbent polymer has a Gel Integrity Index (GII) of less than about 1 kg mm.

69. The method of claim 62, wherein the superabsorbent polymer has a Gel Integrity Index (GII) of less than about 0.05 kg mm.

70. The method of claim 62, wherein the superabsorbent polymer has a Gel Integrity Index (GII) of about 0.10 kg mm to about 0.30 kg mm.

71. A method of preparing an absorbent article comprising:
combining a superabsorbent polymer having a Gel Integrity Index of less than about 500 kg mm with wettable fibers to form an absorbent core and disposing the absorbent core between a substantially impermeable backsheet and a permeable topsheet.

72. The method of claim 71, wherein the superabsorbent polymer is about 10% to about 80% by weight of the absorbent core.

73. The method of claim 71, wherein the superabsorbent polymer is about 20% to about 60% by weight of the composition.

74. The method of claim 71, wherein the superabsorbent polymer is about 30% to about 50% by weight of the composition.

75. The method of claim 71, wherein the wettable fibers comprise about 20% to about 90% of the composition.

76. The method of claim 71, wherein the wettable fibers comprise about 50% to about 70% by weight of the composition.

77. The method of claim 71, wherein the superabsorbent polymer has an AUL value of less than about 25 g/g.

78. The method of claim 71, wherein the superabsorbent polymer has a Gel Integrity Index (GII) of less than about 10 kg mm.

79. The method of claim 71, wherein the superabsorbent polymer has a Gel Integrity Index (GII) of less than about 1 kg mm.

80. The method of claim 71, wherein the superabsorbent polymer has a Gel Integrity Index (GII) of less than about 0.05 kg mm.

81. The method of claim 71, wherein the superabsorbent polymer has a Gel Integrity Index (GII) of about 0.10 kg mm to about 0.30 kg mm.
